(19) United States
(12) Patent Application Publication
(10) Pub. No.: US 2017/0151959 A1
(43) Pub. Date: Jun. 1, 2017

(54) Autonomous vehicle with interactions with wearable devices

(71) Applicant: BRAGI GmbH, Munchen (DE)

(72) Inventor: Peter Vincent Boesen, Munchen (DE)

(21) Appl. No.: 15/357,063

(22) Filed: Nov. 21, 2016

Related U.S. Application Data
(60) Provisional application No. 62/260,445, filed on Nov. 27, 2015.

(51) Int. Cl. B60W 50/12; B60W 40/08; G01C 21/36; G05D 1/00

(52) U.S. Cl. CPC B60W 50/12; G05D 1/0061; B60W 40/08; G01C 21/3617; B60W 2040/0836; B60W 2040/0872

(57) ABSTRACT

A system includes an vehicle having functionality for autonomous operation, a vehicle network disposed within the vehicle, and an earpiece comprising an earpiece housing, a physiological monitoring sensor, an intelligent control system operatively connected to the physiological monitoring sensor and disposed within the ear piece housing, and a wireless transceiver disposed within the earpiece housing and operatively connected to the intelligent control system. The vehicle is configured to receive health data from the ear piece and in response to the health data perform one or more functions independent of a vehicle occupant using the earpiece.

### PRIORITY STATEMENT

This application claims priority to U.S. Provisional Patent Application 62/260,445, filed on Nov. 27, 2015, and entitled Autonomous vehicle with interactions with wearable devices, hereby incorporated by reference in its entirety.

### FIELD OF THE INVENTION

The present invention relates to wearable devices. More particularly, but not exclusively, the present invention relates to autonomous vehicles using wearable devices.

### BACKGROUND

Autonomous or self-driving vehicles provide the potential for improving vehicle safety, providing for more orderly traffic patterns, and allowing individuals to recapture commuting time by allowing for the potential to perform other activities while “driving.” What is needed are autonomous vehicles with improved electronics options which create, improve, or enhance safety or overall experience of autonomous vehicles. In particular, what is needed are autonomous vehicles which integrate with wearable devices.

### SUMMARY

Therefore, it is a primary object, feature, or advantage of the present invention to improve over the state of the art.

It is another object, feature, or advantage of the present invention to communicate between vehicle systems and wearable devices.

It is a further object, feature, or advantage of the present invention to use wearable devices within autonomous vehicles and to provide enhanced vehicle functionality.

It is another object, feature, or advantage of the present invention to enhance the safety of an autonomous vehicle using wearable devices.

One or more of these and or other objects, features, or advantages of the present invention will become apparent from the specification and claims that follow. No single embodiment need provide each and every object, feature, or advantage. Different embodiments may have different objects, features, or advantages. Therefore, the present invention is not to be limited to or by an objects, features, or advantages stated herein.

According to one aspect a system includes an vehicle having functionality for autonomous operation, a vehicle network disposed within the vehicle and an earpiece comprising an earpiece housing, a physiological monitoring sensor, an intelligent control system such as may include a processor operatively connected to the physiological monitoring sensor and disposed within the ear piece housing, and a wireless transceiver disposed within the earpiece housing and operatively connected to the intelligent control system. The vehicle is configured to receive health data from the ear piece and in response to the health data perform one or more functions independent of a vehicle occupant using the earpiece. The physiological monitoring sensor may be an inertial sensor, a glucose sensor, an alcohol sensor, a temperature sensor, or a pulse oximeter or other type of sensor. The vehicle may be configured to determine presence of a health condition based on the health data and lock vehicle controls to prevent the vehicle occupant from operating the vehicle controls or to change destination settings to a nearest emergency room or pull over and place a call to an emergency responder.

According to another aspect a method includes sensing physiological data at one or more physiological sensors of an ear piece of an occupant of a self-driving vehicle, wirelessly communicating a representation of the physiological data from the ear piece to a vehicle network of the self-driving vehicle, and performing an action by the self-driving vehicle in response to the physiological data and independently from the occupant to enhance safety of the self-driving vehicle. The physiological data may include pulse oximeter data, inertial sensor data, temperature data, glucose sensor data, or other data. The action may be to lock driver controls to prevent the occupant from over-riding autonomous operation or other action or actions.

### BRIEF DESCRIPTION OF THE DRAWINGS

FIG. 1 illustrates one example of use of a wearable device in conjunction with a vehicle.

FIG. 2 illustrates a wearable device in the form of a set of ear pieces.

FIG. 3 is a block diagram illustrating a device.

FIG. 4 illustrates a system which includes ear pieces in communication with a vehicle.

FIG. 5 illustrates a wearable device in communication with various vehicle systems through a vehicle network.

### DETAILED DESCRIPTION

Some of the most important factors in selecting a vehicle such as car may be the technology available to enhance the experience. This may be of particular importance in certain vehicle segments such as for luxury vehicles. Another important factor in selecting a vehicle may be the available safety features. According to various aspects, the present invention allows for wearable devices such as earpieces to enhance the overall safety of the vehicle. Therefore, it is expected that the technology described herein will make any vehicle so equipped more desirable to customers, more satisfying to customers, and potentially more profitable for the vehicle manufacturer. Similarly at least some of the various aspects may be added to existing vehicles as after-market accessories to improve the safety or experience of existing vehicles.

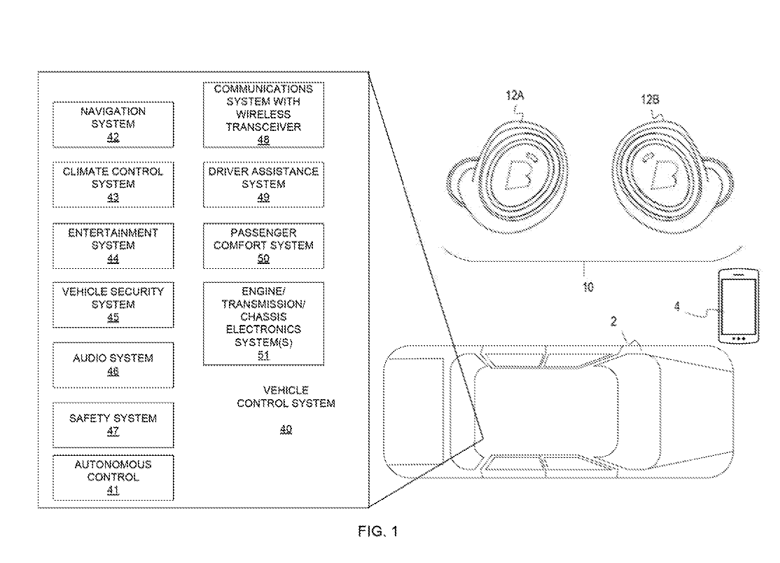

FIG. 1 illustrates one example of use of a wearable device in conjunction with a vehicle. A shown in FIG. 1 there is a vehicle 2. Although the vehicle shown is a full-size sedan, it is contemplated that the vehicle may be of any number of types of cars, trucks, sport utility vehicles, vans, mini-vans, automotive vehicles, commercial vehicles, agricultural vehicles, construction vehicles, specialty vehicles, recreational vehicles, buses, motorcycles, aircraft, boats, ships, yachts, spacecraft, or other types of vehicles. The vehicle may be gas-powered, diesel powered, electric, solar-powered, or human-powered. The vehicle may be actively operated by a driver or may be partially or completely autonomous or self-driving. The vehicle 2 may have a vehicle control system 40. The vehicle control system is a system which may include any number of mechanical, electrical, and electromechanical subsystems. As shown in FIG. 1, such systems may include a self-driving or autonomous control system 41, a navigation system 42, an entertainment system 44, a vehicle security system 45, an audio system 46, a safety system 47, a communications system 48 preferably with a wireless transceiver, a driver assistance system 49, a passenger comfort system 50, and an engine/transmission, chassis electronics system(s) 51. Of course, other examples of vehicle control sub-systems are contemplated. In addition, it is to be understood that there may be overlap between some of these different vehicle systems and the presence or absence of these vehicle systems as well as other vehicle systems may depend upon the type of vehicle, the type of fuel or propulsion system, the size of the vehicle, and other factors and variables. In the automotive context, examples of the driver assistance system 49 may include one or more subsystems such as a lane assist system, a speed assist system, a blind spot detection system, a park assist system, and an adaptive cruise control system. In the automotive context, examples of the passenger comfort system 50 may include one or more subsystems such as automatic climate control, electronic seat adjustment, automatic wipers, automatic headlamps, and automatic cooling. In the automotive context, examples of the safety system 47 may include active safety systems such as air bags, hill descent control, and an emergency brake assist system. Aspects of the navigation system 42, the entertainment system 44, the audio system 46, and the communications system 48 may be combined into an infotainment system.

One or more wearable devices such as a set of earpieces 10 including a left earpiece 12A and a right earpiece 12B may be in operative communication with the vehicle control system 40 such as through the communication system 48. For example, the communication system 48 may provide a Bluetooth or BLE link or Wi-Fi link to wearable devices or may otherwise provide for communications with the wearable devices preferably through wireless communications. The vehicle 2 may communicate with the wearable device(s) directly, or alternatively, or in addition, the vehicle 2 may communicate with the wearable device(s) through an intermediary device such as a mobile device 4 which may be a mobile phone, a tablet, or other type of mobile device or computing device.

As will be explained in further details with respect to various examples, the wearable device(s) 10 interact with the vehicle control system 40 in any number of different ways. For example, the wearable device(s) 10 may provide sensor data, identity information, stored information, streamed information, or other types of information to the vehicle. Based on this information, the vehicle may take any number of actions which may include one or more actions taken by the vehicle control system (or subsystems thereof). In addition, the vehicle 2 may communicate sensor data, identity information, stored information, streamed information or other types of information to the wearable device(s) 10.

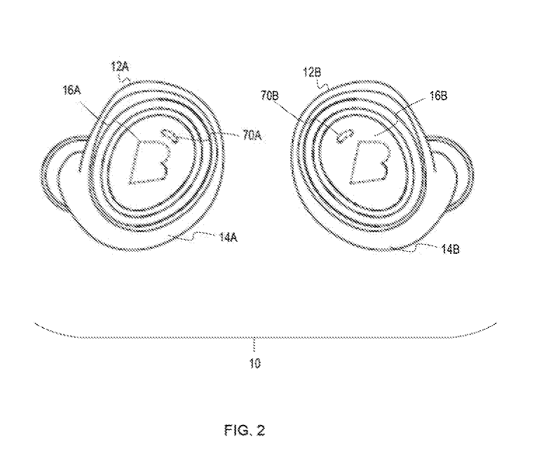

FIG. 2 illustrates one example of a wearable device in the form of a set of ear pieces 10 in greater detail. FIG. 1 illustrates a set of earpiece wearables 10 which includes a left earpiece 12A and a right earpiece 12B. Each of the earpieces wearables 12A, 12B has an earpiece wearable housing 14A, 14B which may be in the form of a protective shell or casing and may be an in-the-ear earpiece housing. A left infrared through ultraviolet spectrometer 16A and right infrared through ultraviolet spectrometer 16B is also shown. Each earpiece 12A, 12B may include one or more microphones 70A, 70B. Note that the air microphones 70A, 70B are outward facing such that the air microphones 70A, 70B may capture ambient environmental sound. It is to be understood that any number of microphones may be present including air conduction microphones, bone conduction microphones, or other audio sensors.

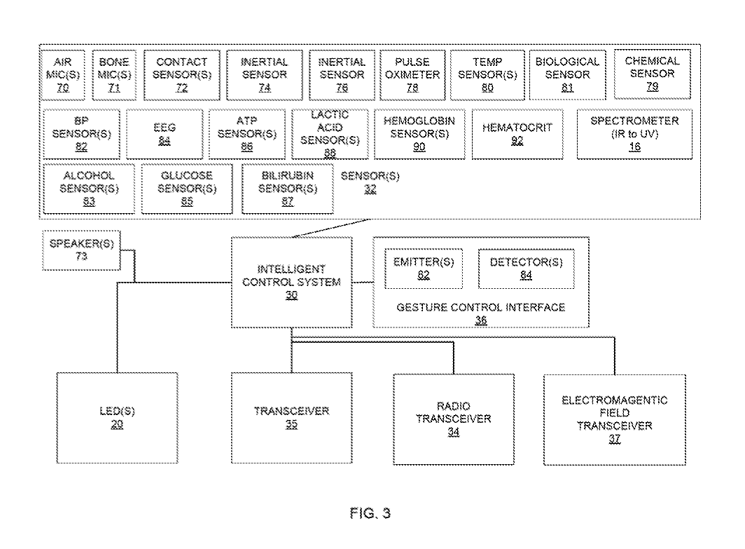

FIG. 3 is a block diagram illustrating a device. The device may include one or more LEDs 20 electrically connected to an intelligent control system 30. The intelligent control system 30 may include one or more processors, microcontrollers, application specific integrated circuits, or other types of integrated circuits. The intelligent control system 30 may also be electrically connected to one or more sensors 32. Where the device is an earpiece, the sensor(s) may include an inertial sensor 74, another inertial sensor 76. Each inertial sensor 74, 76 may include an accelerometer, a gyro sensor or gyrometer, a magnetometer or other type of inertial sensor. The sensor(s) 32 may also include one or more contact sensors 72, one or more bone conduction microphones 71, one or more air conduction microphones 70, one or more chemical sensors 79, a pulse oximeter 76, a temperature sensor 80, or other physiological or biological sensor(s). Further examples of physiological or biological sensors include an alcohol sensor 83, glucose sensor 85, or bilirubin sensor 87. Other examples of physiological or biological sensors may also be included in the device. These may include a blood pressure sensor 82, an electroencephalogram (EEG) 84, an Adenosine Triphosphate (ATP) sensor, a lactic acid sensor 88, a hemoglobin sensor 90, a hematocrit sensor 92 or other biological or chemical sensor.

A spectrometer 16 is also shown. The spectrometer 16 may be an infrared (IR) through ultraviolet (UV) spectrometer although it is contemplated that any number of wavelengths in the infrared, visible, or ultraviolet spectrums may be detected. The spectrometer 16 is preferably adapted to measure environmental wavelengths for analysis and recommendations and thus preferably is located on or at the external facing side of the device.

A gesture control interface 36 is also operatively connected to or integrated into the intelligent control system 30. The gesture control interface 36 may include one or more emitters 82 and one or more detectors 84 for sensing user gestures. The emitters may be of any number of types including infrared LEDs. The device may include a transceiver 35 which may allow for induction transmissions such as through near field magnetic induction. A short range transceiver 34 using Bluetooth, BLE, UWB, or other means of radio communication may also be present. The short range transceiver 34 may be used to communicate with the vehicle control system. In operation, the intelligent control system 30 may be configured to convey different information using one or more of the LED(s) 20 based on context or mode of operation of the device. The various sensors 32, the processor 30, and other electronic components may be located on the printed circuit board of the device. One or more speakers 73 may also be operatively connected to the intelligent control system 30.

An electromagnetic (E/M) field transceiver 37 or other type of electromagnetic field receiver is also operatively connected to the intelligent control system 30 to link the processor 30 to the electromagnetic field of the user. The use of the E/M transceiver 37 allows the device to link electromagnetically into a personal area network or body area network or other device.

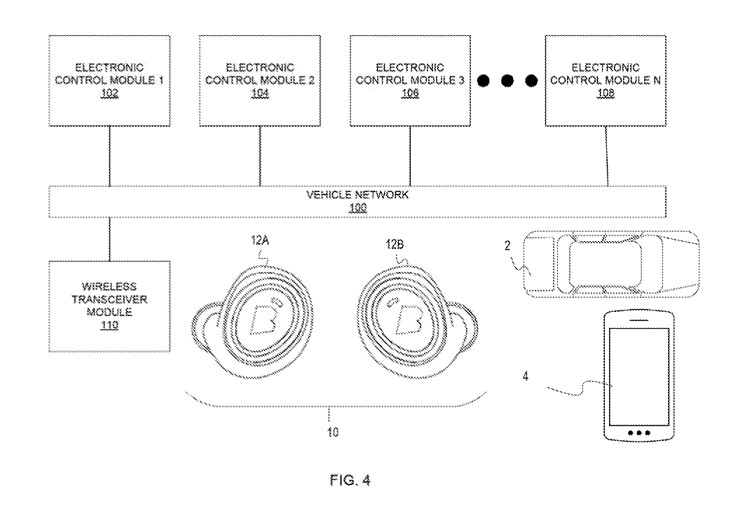

FIG. 4 illustrates another example of one or more wearable ear pieces 10 in operative communication with a vehicle 2. In FIG. 4, a vehicle network 100 is shown. According to one aspect, the wearable devices 12A, 12B may communicate information through a vehicle network 100 associated with a vehicle 2. Data, instructions, alerts, or other information may be communicated over the vehicle network 100 or vehicle bus to and from the wearable devices. Protocols which are used may include a Controller Area Network (CAN), Local Interconnect Network (LIN), or others including proprietary network protocols or network protocol overlays.

Various types of electronic control modules 102, 104, 106, 108 or electronic control units may communicate over the network 100 of the vehicle. These may include electronic modules such as an engine control unit (ECU), a transmission control unit (TCU), an anti-lock braking system (ABS), a body control module (BCM), a door control unit (DCU), an electric power steering control unit (PSCU), a human-machine interface (HMI), powertrain control module (PCM), speed control unit (SCU), telematic control unit (TCU), brake control unit (BCM), battery management system, vehicle navigation system, entertainment system, infotainment system, and numerous others. Any number of electronic control modules may be operatively connected to the vehicle network 100.

In one embodiment a wireless transceiver module 110 is operatively connected to a vehicle network 100 and it is the wireless transceiver module 110 which is in operative communication with one or more wearable devices such as wearable ear piece 12A, 12B.

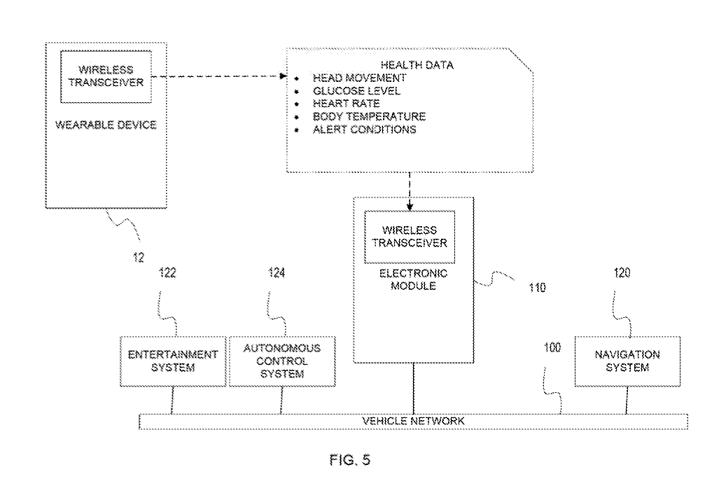

As shown in FIG. 5, one or more wearable devices 12 (including one or more ear pieces from one or more different vehicle occupants) may communicate with a navigation system 120 of a vehicle. Although the communication may be performed directly between one or more systems of the vehicle and one or more ear pieces 12, in one embodiment a wireless transceiver module 110 may be operatively connected to the wearable ear piece 12 after the transceiver module 110 connects with or forms a wireless linkage with one or more of the ear pieces 12. The wireless transceiver module 110 may use any number of different types of communications and protocols including Bluetooth, Bluetooth Low Energy (BLE), ultra-wideband, Wi-Fi, or otherwise. The vehicle network 100 may provide for communicating with any number of different modules or systems including a navigation system 120 and an entertainment system 122. The vehicle systems or modules may further include an autonomous control system 124 which is used for autonomous or self-driving of the vehicle.

According to another aspect, one or more wearable devices may provide for health monitoring of an individual such as a driver or passenger of the vehicle. The wearable devices may have any number of different sensors which may be used for monitoring the health of an individual or other physical parameters of an individual. Examples of sensors may include one or more inertial sensors such as an accelerometer, a gyro sensor or gyrometer, a magnetometer or other type of inertial sensor. As shown in FIG. 3, the sensor(s) 32 may also include one or more contact sensors 72, one or more bone conduction microphones 71, one or more air conduction microphones 70, one or more chemical sensors 79, a pulse oximeter 78, a temperature sensor 80, or other physiological or biological sensor(s). Further examples of physiological or biological sensors include an alcohol sensor 83, glucose sensor 85, or bilirubin sensor 87. Other examples of physiological or biological sensors may also be included in the device. These may include a blood pressure sensor 82, an electroencephalogram (EEC) 84, an Adenosine Triphosphate (ATP) sensor, a lactic acid sensor 88, a hemoglobin sensor 90, a hematocrit sensor 92 or other biological or chemical sensor.

These various sensors may be used in any number of ways to provide feedback to the vehicle. For example, the wearable devices may be used to detect emergency conditions associated with an occupant of the vehicle. Where the wearable device is an earpiece, the inertial sensors may be used to track head movement of a vehicle occupant. If the head movement of the occupant is indicative that the user is falling asleep, such as downward movement of the chin and then snapping back of the head as the user catches themselves falling asleep, or other movements associated with a user falling asleep, then the earpiece may communicate a message to the vehicle. Upon receipt of the message, the vehicle may take any number of relevant actions. This may include, turning on autonomous or self driving operation if this feature is not already turned on, locking out the user from manual control or locking out the ability to override autonomous control. It may include locating the nearest rest stop or hotel or motel and navigating to it, changing destination to the nearest hospital or emergency room, pulling over to the side of the road or at a next exit, providing one or more audio warnings, placing a phone call or any number of other actions.

Another example of use of a sensor is use of a glucose sensor. If the blood sugar of an individual is low as measured with a wearable device, the wearable device may communicate a message to the vehicle. Upon receipt of the message, the vehicle may take any number of relevant actions. This may include locating the nearest rest stop, restaurant, or gas station so that the individual may obtain something to eat, providing an audio message such as reminding the user to eat something, alerting occupants within the vehicle, turning, on an autonomous mode and locking out the occupant from manual override and navigating to the nearest place where food is likely to be available, or any number of other actions.

Another example of use of a sensor is use of an alcohol sensor. If the wearable device detects that the driver may be impaired based upon alcohol levels, then the wearable device may communicate an appropriate message to the vehicle which may disable its operation, place it in an autonomous driving only mode so that the occupant cannot override the vehicle, provide an audio message, make a phone call, or perform any number of other actions.

Yet another example of use of a sensor is a pulse oximeter. If the wearable device detects that the driver heart rate of the driver is increasing then appropriate action may be taken. For example, if the wearable device detects heart rate indicative of a heart attack or other serious condition, the autonomous vehicle may drive to the nearest emergency room, place a call to an emergency responder and pull over to the side of the road or safe location, or take other appropriate actions.

The various sensors may be used in any number of other ways including detecting health status or predicting health status which may be indicative of a health condition or event which may impair safe driving. Where the heath condition or event may impair safe driving, the autonomous vehicle may lock vehicle controls so that the occupant cannot override autonomous vehicle functions. If the occupant is driving, then the autonomous vehicle may take over control from the occupant immediately.

Returning to FIG. 5, various types of health data may be communicated to the vehicle including, without limitation, head movement, glucose levels, heart rate, and body temperature. In addition, one or more alert conditions may be communicated to the vehicle as well. Based on the health data and/or the alert condition(s) the self-driving, or autonomous vehicle may then perform the appropriate action in response to the condition as previously described.

Various methods, system, and apparatus have been shown and described relating to vehicles with wearable integration or communication. The present invention is not to be limited to these specific examples but contemplates any number of related methods, system, and apparatus and these examples may vary based on the specific type of vehicle, the specific type of wearable device, the various types of health conditions and health data, the alert conditions where present, and the actions taken in response to health data and other considerations.

What is claimed is:

1. A system comprising:
an vehicle having functionality for autonomous operation;
a vehicle network disposed within the vehicle;
an earpiece comprising an earpiece housing, a physiological monitoring sensor, an intelligent control system operatively connected to the physiological monitoring sensor and disposed within the ear piece housing, and a wireless transceiver disposed within the earpiece housing and operatively connected to the intelligent control system;
wherein the vehicle is configured to receive health data from the ear piece and in response to the health data perform one or more functions independent of a vehicle occupant using the earpiece.

2. The system of claim 1 wherein the physiological monitoring sensor is an inertial sensor.

3. The system of claim 1 wherein the physiological monitoring sensor is a glucose sensor.

4. The system of claim 1 wherein the physiological monitoring, sensor is an alcohol sensor.

5. The system of claim 1 wherein the physiological monitoring sensor is a temperature sensor.

6. The system of claim 1 wherein the physiological monitoring sensor is a pulse oximeter.

7. The system of claim 1 wherein the vehicle is configured to determine presence of a health condition based on the health data and lock vehicle controls to prevent the vehicle occupant from operating the vehicle controls.

8. The system of claim 1 wherein the vehicle is configured to determine presence of a health condition based on the health data and change destination settings to a nearest emergency room.

9. The system of claim 1 wherein the vehicle is configured to determine presence of a health condition based on the health data and change destination settings to a nearest hospital.

10. The system of claim 1 wherein the vehicle is configured to determine presence of a health condition based on the health data and pull over and place a call to an emergency responder.

11. A method comprising:
sensing physiological data at one or more physiological sensors of an ear piece of an occupant of a self-driving vehicle;
wirelessly communicating a representation of the physiological data from the ear piece to vehicle network of the self-driving vehicle;
performing an action by the self-driving vehicle in response the physiological data and independently from the occupant to enhance safety of the self-driving vehicle.

12. The method of claim 11 wherein the physiological data comprises pulse oximeter data.

13. The method of claim 12 wherein the physiological data further comprises inertial sensor data.

14. The method of claim 12 wherein the physiological data further comprises temperature data.

15. The method of claim 12 wherein the physiological data further comprises glucose sensor data.

16. The method of claim 11 wherein the action is to lock driver controls to prevent the occupant from over-riding autonomous operation.
